(19) United States
(12) Patent Application Publication
(10) Pub. No.: US 2025/0253028 A1
(43) Pub. Date: Aug. 7, 2025

(54) System For Focused Microwave Transmission

(71) Applicant: The Regents of the University of Colorado, a body corporate, Denver, CO (US)

(72) Inventors: Vijay Harid, Englewood, CO (US); Tim Lei, Thornton, CO (US); Hoyoung Kim, Brookline, MA (US); Ben-Zheng Li, Aurora, CO (US)

(21) Appl. No.: 19/047,829

(22) Filed: Feb. 7, 2025

Related U.S. Application Data
(60) Provisional application No. 63/550,866, filed on Feb. 7, 2024.

(51) Int. Cl. G16H 20/40; A61N 5/02; A61N 5/04; G16H 30/20

(52) U.S. Cl. CPC G16H 20/40; G16H 30/20; A61N 5/025; A61N 5/045

(57) ABSTRACT

Technology is disclosed herein for a system, devices, and methods for microwave focusing in various implementations. In an implementation, a computing device identifies a location for receiving focused microwave emissions from an array of antennas. The computing device determines an optimal antenna configuration for illuminating the location by the microwave emissions based at least on a configuration of the array and causes the antennas to illuminate the location according to the optimal antenna configuration. In an implementation, the computing device determines the optimal antenna configuration for illuminating the location based on a spatial distribution of material properties of a volume which includes the location. In an implementation, the spatial distribution is based on imaging data of the volume. In various implementations, the configuration of the array includes a spherical or cylindrical distribution of antenna positions.

### INSERT CROSS REFERENCE TO RELATED APPLICATIONS

This application is related to and claims the benefit of priority to U.S. Provisional Patent Application No. 63/550,866 entitled “SYSTEM FOR FOCUSED MICROWAVE TRANSMISSION,” filed Feb. 7, 2024, the contents of which are incorporated by reference in their entirety for all purposes.

### TECHNICAL FIELD

Aspects of the disclosure are related to the field of controlling microwave transmission for therapeutic use.

### BACKGROUND

Minimally invasive therapeutic procedures involve medical interventions that utilize advanced technologies and techniques to treat various conditions with reduced impact on the patient's body compared to traditional surgical methods. These procedures typically utilize small incisions, specialized instruments, and imaging guidance, aiming to minimize trauma, scarring, and recovery time. For example, high frequency electromagnetic waves, such as microwaves, can be used for tissue stimulation or ablation via conductive heating but require surgical insertion of a microwave antenna. While minimally invasive therapies often offer benefits such as quicker recovery and lower infection rates, potential risks may include complications related to anesthesia, bleeding, or damage to surrounding tissues.

In contrast to invasive and minimally invasive medical treatment, non-invasive medical treatments do not require penetrating the skin or disrupting bodily tissues. Non-invasive procedures are designed to minimize patient discomfort, reduce recovery time, and lower the risk of complications associated with invasive methods. While non-invasive treatments generally offer advantages such as decreased recovery periods and lower infection risks, potential drawbacks may include limited effectiveness for certain conditions and the necessity for ongoing or repeated treatments. For example, Transcranial Magnetic Stimulation employs low frequency magnetic fields to alleviate symptoms of neurologic disorders through neural stimulation. However, spatial focusing of low frequency waves for magnetic stimulation is difficult and requires large and complex coil designs. Alternatively, low frequency electric fields can be used to modulate neuron firing but require the probe to have good contact with the scalp to drive the current, which may be inconvenient in certain applications. Ultrasound can be used to locally modulate neural activity, but because ultrasound energy is highly absorptive to bone, such treatment requires high power which runs the risk of localized heating.

### OVERVIEW

Technology is disclosed herein for a system, devices, and methods for microwave focusing in various implementations. In an implementation, a computing device identifies a location for receiving focused microwave emissions from an array of antennas. The computing device determines an optimal antenna configuration for illuminating the location by the microwave emissions based at least on a configuration of the array and causes the antennas to illuminate the location according to the optimal antenna configuration. In an implementation, the optimal antenna configuration includes emission parameters and orientation information for each antenna of the array.

In an implementation, the computing device determines the optimal antenna configuration for illuminating the location based on a spatial distribution of material properties of a volume which includes the location. In an implementation, the spatial distribution is based on imaging data of the volume. In various implementations, the configuration of the array includes a spherical or cylindrical distribution of antenna positions.

This Overview is provided to introduce a selection of concepts in a simplified form that are further described below in the Detailed Description. It may be understood that this Overview is not intended to identify key features or essential features of the claimed subject matter, nor is it intended to be used to limit the scope of the claimed subject matter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an operational environment for microwave focusing in an implementation.

FIG. 2 illustrates a process for microwave focusing in an implementation.

FIG. 3 illustrates an operational scenario for microwave focusing in an implementation.

FIG. 4 illustrates an operational scenario for microwave focusing in an implementation.

FIGS. 5A and 5B illustrate operational scenarios for microwave focusing in an implementation.

FIG. 6 illustrates a computing system suitable for implementing the various operational environments, architectures, processes, scenarios, and sequences discussed below with respect to the other Figures.

### DETAILED DESCRIPTION

Various implementations are disclosed herein for a system to focus microwave emissions to produce a localized hot spot with minimal or no line-of-sight illumination. The system may be used for non-invasive and non-destructive treatment of biological tissue by finely focusing microwave energy to stimulate and/or ablate biological tissue with minimal or no effect on surrounding tissue.

In an implementation, a three-dimensional array of individually controlled microwave antennas is tuned to focus microwave signals to create sub-centimeter-scale spot sizes which may be used for biomedical as well as non-medical applications. The geometry of the three-dimensional array may be spherical or cylindrical such that the array surrounds the body part to be treated. For example, for neurological treatment of the brain, a spherical arrangement of dipoles in a helmet may be used. By carefully configuring the emission parameters (e.g., frequency, amplitude including amplitude modulation, phase, and polarization) and orientation of the antennas, the system drives the antennas to generate, through constructive interference, a microwave hot spot at a desired location while destructive interference reduces or eliminates sidelobes. Moreover, by using a dispersed array of transmitters rather than a concentrated microwave beam, illumination of non-target tissue along the line-of-sight of the beam is avoided. Thus, treatment can be isolated to a specific three-dimensional region or hot spot while minimally affecting the surrounding area.

In an implementation, the system, comprising multiple transmitting dipoles dispersed in a spherical or cylindrical shell configuration, modulates the amplitude, phase, and polarity of the dipoles according to an optimization algorithm to achieve a desired electric or magnetic field profile in three dimensions. Using the optimization algorithm, the system determines a transmitter strength for each dipole to generate a prescribed hot spot profile at a given location in the biological tissue, e.g., in the brain. By changing the dipole configurations, the system can steer the hot spot or focal point to a precise location. Indeed, the system can be enabled to perform a microwave scan of the biological tissue (e.g., a limb) by rapidly repositioning the microwave hot spot by changing the emission parameters of the dipole antennas in the dipole array. The ability to rapidly reposition the microwave hot spot by adjusting the emission parameters can be used for treatment of multiple locations in the biological tissue. Moreover, the ability to scan the biological tissue can be used for imaging or mapping the tissue.

The number of dipoles may be selected according to how tightly the microwave fields are to be focused and a need for optimal interference patterns (e.g., such as to reduce undesired sidelobes outside the target area). For example, a spherical shell configuration for a helmet-type device may include an array of 16, 32, or 64 dipoles, where a greater number of dipoles corresponds to a tighter focus and fewer sidelobes through destructive interference. In various implementations, the microwave frequency is a value in the range of 100 MHz to 10 GHz which allows the microwaves to penetrate to a depth of 10 cm to 30 cm depending on the medium (e.g., biological tissue to be treated), with higher frequencies penetrating farther. Further, by adjusting the transmission strength of the microwaves, the system can be used for neural stimulation (i.e., modulation of neural activity) or for ablation. To modulate or stimulate the neural activity, in an implementation, amplitude modulation (e.g., a sinusoidal signal) is superimposed on the microwave signal to increase or to decrease firing rates of neural spikes. Targeting the tissue with low-power microwaves, but to which a sinusoidal signal (e.g., a 100 Hz sinusoidal signal) is superimposed, can cause neural stimulation or inhibition while avoiding burning the tissue.

To tune the array of dipole antennas to target a desired location in a medium, the system computes an optimization algorithm which determines the magnitudes and orientations of the multiple transmitting dipoles based on a desired electric field profile and a spatial distribution profile of the dielectric permittivity and electrical conductivity of the medium (e.g., the body part to be treated) and a peak energy at the desired location. For example, to treat a location in the brain, the dielectric and conductivity distribution profile may be based on a skull geometry which includes a volume of brain material sheathed in a layer of bone material and an outer layer of skin material. In an implementation, the algorithm numerically computes a baseline or model electric field by summing the electric field contributions of the dipoles at grid point of a three-dimensional orthogonal grid which contains the dipole array surrounding the skull geometry. The algorithm then reduces the regularization error between the model electric field and the desired electric field. The algorithm generates a unique solution describing the emission parameters (e.g., amplitude, phase, polarity) and pointing directions of the dipole antennas to achieve the desired electric field profile.

In various implementations, three-dimensional profiles of dielectric permittivity and electrical conductivity for a body part to be treated can be generated based on imaging the body part, such as magnetic resonance imaging (MRI) or computed tomography (CT) scans, then fed to the optimization algorithm to compute the emission parameters and orientations of the individual dipole antennas.

Tuning the array of dipole antennas based on the results of the optimization algorithm to obtain the optimal interference pattern can be controlled by a controller using techniques such as phase shifting, beam switching, or beam steering.

Technical effects of the technology disclosed herein allow for non-invasive, high-precision therapies of disease including neural stimulation and neural ablation. By configuring the multiple microwave antennas in terms of direction and signal strength, the microwaves can be targeted to the treatment area (for example, on the order of millimeters) while avoiding side effects to the surrounding tissue. Amplitude modulation of the microwave emissions can be used to stimulate neural tissue at a low power which mitigates the risk of tissue damage. The focused microwaves at higher power levels can be used to lesion tissue. In some implementations, multiple neural regions may be treated simultaneously based on rapidly repositioning the microwave focus by electronically-controlled adjustment of the amplitude and phases of the microwave fields. Similarly, in some implementations, the technology can be used to scan biological tissue to generate high-resolution images of the tissue.

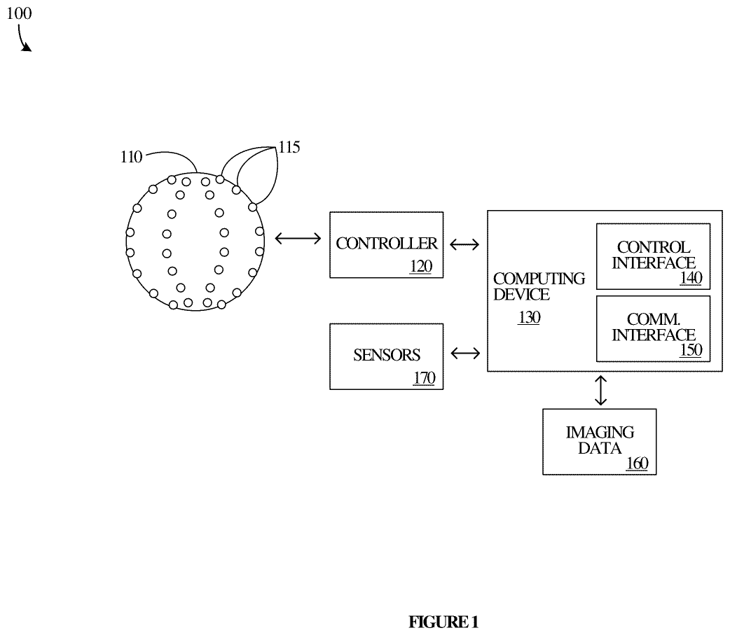

Turning to the Figures, FIG. 1 illustrates operational architecture 100 for a system for focused microwave transmission in an implementation. Operational architecture 100 includes device 110 comprising microwave dipole antennas 115 which are controlled by controller 120. Operational architecture 100 also includes computing device 130 which includes control interface 140 and communications interface 150. Operational architecture also includes imaging data 160 used by computing device 130 for treatment and sensors 170 which provides sensor data to computing device 110.

Device 110 is representative of a device including an array of antennas 115 for transmitting microwave signals. In an implementation, device 110 is a helmet-type device with antennas 115 arranged in a spherical configuration which is enclosed around the patient's head for treatment of neural cells of the brain. Although illustrated as a spherical geometry, device 110 is also representative of devices which hold antennas 115 in other configurations, such as a cylindrical configuration for the treatment of an arm or leg.

Antennas 115 are representative of microwave dipole antennas for transmitting electromagnetic energy in a microwave frequency range, such as helical coil antennas. Antennas 115 may be positioned at a fixed location on device 110 with a wired connection to controller 120. In operation, each of antennas 115 is individually controlled by controller 120 to emit a desired microwave frequency according to emission parameters (e.g., amplitude, phase, polarization) determined according to an optimization algorithm for focused microwave transmission.

Controller 120 is representative of a computing device for controlling antennas 115, including frequency, amplitude, phase, polarization, and amplitude modulation. In an implementation, controller 120 executes antenna driver software by which to control antennas 115 according to phased array, beam switching, or beam steering technology.

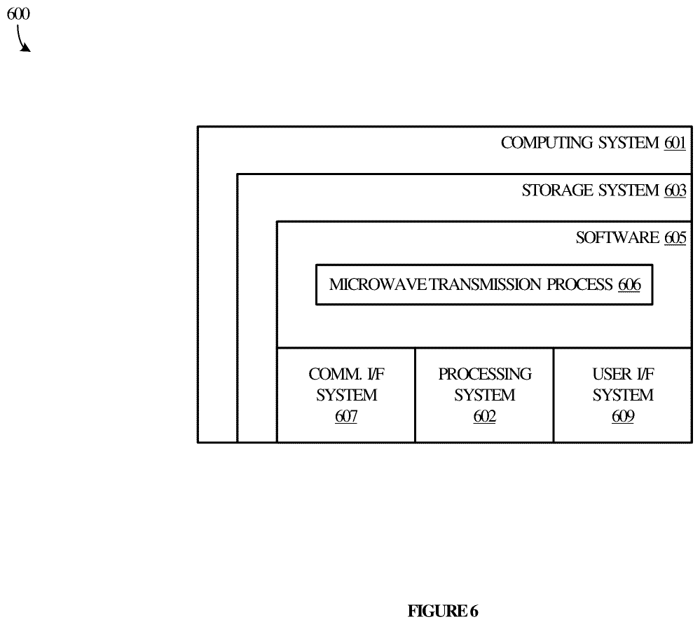

Computing device 130 is representative of a computing device for controlling microwave transmission of antennas 115, such as a laptop computer, desktop computer, or mobile computing device (e.g., smartphone), of which computing device 601 of FIG. 6 is representative. Computing device 130 includes control interface 140 comprising a user interface by which the system is controlled by a user. Control interface 140 of computing device 130 includes an interface by which other devices can communicate with computing device 130, such as computing devices for remote monitoring of the system.

Imaging data 160 comprises data by which an optimization algorithm executed by computing device 130 computes a configuration for antennas 115. Imaging data 160 can include MRI data or CT scan data of the body part to be treated.

Sensors 170 includes sensor devices which provide sensor data such as diagnostic data, imaging data, or environmental data for monitoring the performance of the system, health of the patient, or other feedback which is relevant to the operation of the system.

In some implementations, operational architecture 100 may also include a digital signal processor (not shown) by which to receive data from antennas 115, such as for imaging biological tissue using scanning microwave emissions or microwave tomography.

In a brief operational scenario, a user, such as a medical care provider, identifies a location for neural stimulation or ablation in the brain of a patient. The user indicates the location in control interface 140. Computing device 130 hosts an optimization algorithm by which to compute a solution for configuring antennas 115 to focus microwaves on the indicated location. Computing device 130 receives imaging data 160, such as MRI or CT scan data, by which computing device 130 determines a three-dimensional materials profile for the patient's skull. Based on the electrical conductivity and dielectric permittivity information from the materials profile, the treatment location, the position of the antennas relative to the treatment location, and the microwave frequency for treatment, the optimization algorithm computes the solution for configuring antennas to focus microwaves on the location of treatment. The power level of the microwave energy transmitted by antennas 115 may be determined by the type of treatment to be performed, such as neural stimulation or neural ablation.

Computing device 130 directs controller 120 with respect to the frequency, amplitude, phase, polarization, and orientation of antennas 115 according to the solution generated by the optimization algorithm. Device 110 is worn by the patient such that antennas 115 surround the patient's head.

During treatment, controller 120 causes controls antennas 115 individual, causing a microwave hot spot to be generated by constructive interference at the treatment location. As the system is in operation, computing device 130 may receive information from various ones of sensors 170 pertaining to the health of the patient and/or the performance of the system. This information may be displayed at control interface 140.

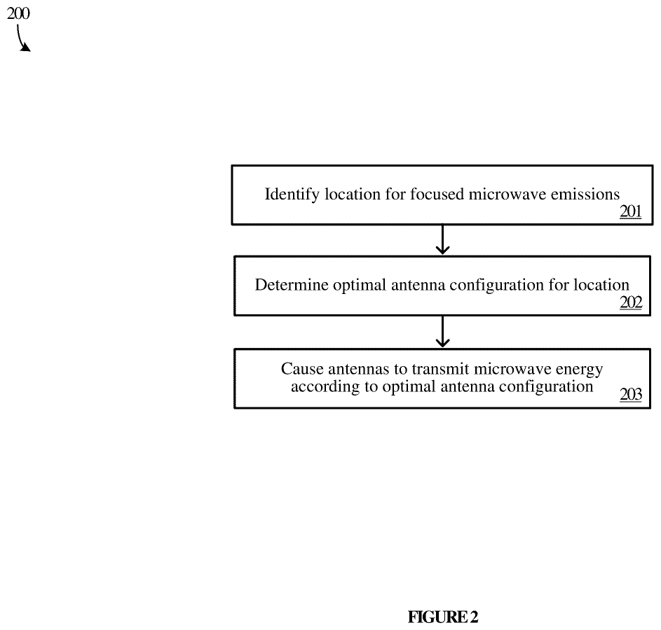

FIG. 2 illustrates a method for microwave focusing in an implementation, herein referred to as process 200. Process 200 may be implemented in program instructions in the context of any of the software applications, modules, components, or other such elements of one or more computing devices. The program instructions direct the computing device(s) to operate as follows, referred to in the singular for the sake of clarity.

A computing device identifies a location for focused microwave energy (step 201). In an implementation, the computing device is connected to an array of microwave dipole antennas which can be individually controlled according to emission parameters of the microwave transmission and an orientation of the antennas. The antennas may be configured in a physical geometry to treat biological tissue such that the antennas positions are dispersed around the tissue and the location to be treated. The location may be indicated as coordinates of three-dimensional stereotactic space or coordinate grid.

In an implementation, the computing device receives imaging data and determines, based on the imaging data, a representation of the biological tissue which includes a spatial distribution of material properties, such as electrical conductivity and dielectric permittivity. Based on the location of treatment and the spatial distribution of material properties, the computing device determines an optimal antenna configuration for the treatment location (step 202). The optimal antenna configuration may include emission parameters such as frequency, amplitude, phase, and polarization for each of the antennas as well as orientation. Emission parameters may also include amplitude modulation of the microwave signal. In an implementation, the computing device determines a solution to an optimization algorithm which receives as input the configuration or position of the antennas relative to the biological tissue to be treated, microwave frequency, the spatial distribution of material properties in the biological tissue, the type of treatment, and the location of treatment.

With the antennas oriented according to the optimal antenna configuration, the computing device causes the antennas to transmit microwave energy according to the optimal antenna configuration (step 203). In various implementations, the computing device causes an antenna controller to control each of the antennas to emit microwaves according to the microwave frequency and operational parameters of the optimal antenna configuration (e.g., emission parameters and antenna orientation). In operation, the antennas emit microwaves such that constructive interference causes a microwave hot spot to be created at the location of treatment at a specified power or energy level which is determined according to the type of treatment. In various implementations, during treatment, the computing device receives sensor data from diagnostic sensors or other types of sensors which provide feedback by which the computing device monitors the health of the patient or the performance of the system.

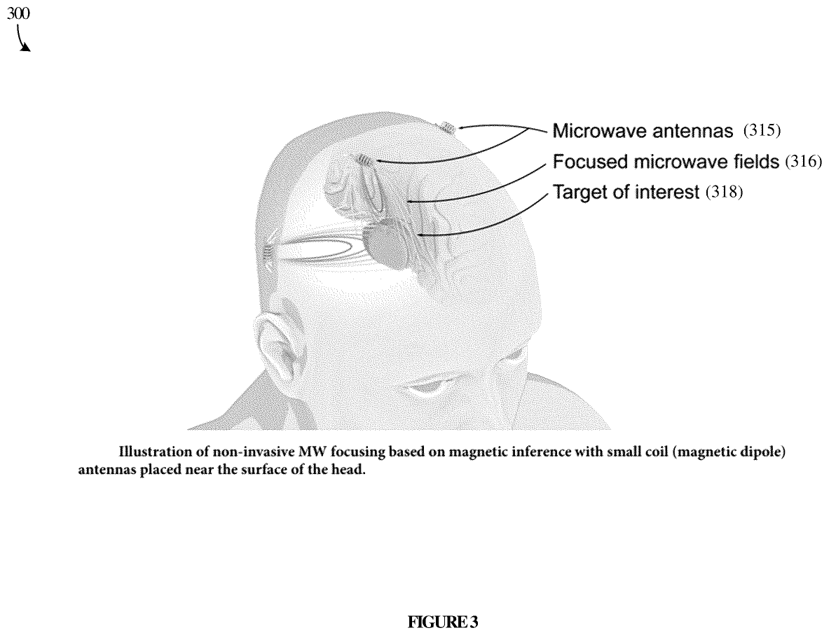

In FIG. 3, operational illustration 300 depicts microwave focusing relating to treatment within the brain in an implementation. In operational illustration 300, a patient receives medical treatment in the form of focused microwaves which cause neural stimulation and/or ablation of cells in the patient's brain. In operational illustration 300, microwave antennas 315, of which antennas 115 are representative, are positioned on the surface of the patient's head. (Although two antennas are depicted, it may be appreciated that applications of the technology disclosed herein can include two or more microwave antennas.) Microwave antennas 315 emit microwaves according to an optimal antenna configuration determined according to optimization algorithm. The optimal antenna configuration specifies the orientation of each antenna along with parameters relating to focusing the microwave transmission of each antenna, such as amplitude, phase, polarization, and frequency. In some scenarios, the optimal antenna configuration also includes amplitude modulation of the microwave transmission, for example, for neural stimulation.

In operation, the microwave fields emitted by antennas 315 generate an area of constructive interference. The optimal antenna configuration causes the constructive interference to occur at target 318 corresponding to the location of treatment. The treatment of the neural tissue at target 318 can include lesioning using high intensity illumination or stimulation of the tissue using lower intensity illumination with amplitude modulation (e.g., a sinusoidal signal superimposed on the microwave carrier signal). In some implementations, focused microwave fields 316 can be repositioned during treatment to treat multiple target locations or to generate imaging data of the tissue by scanning the tissue.

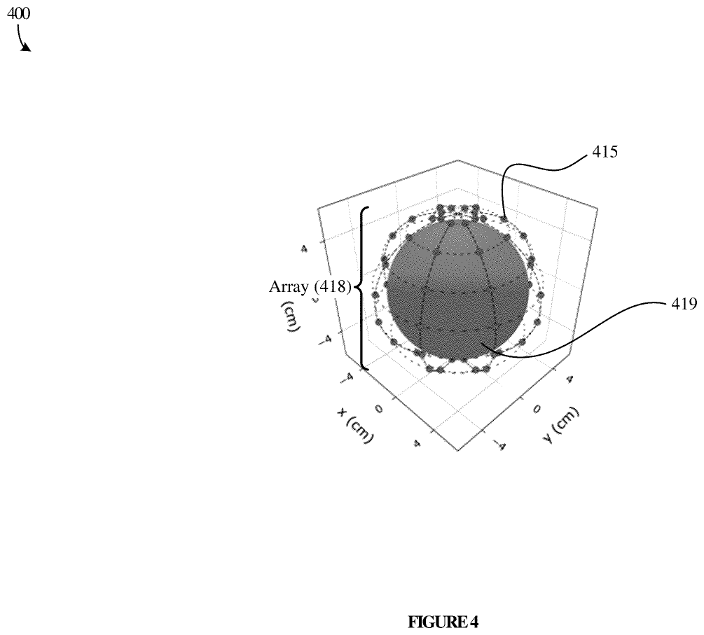

FIG. 4 illustrates operational scenario 400 three-dimensional array 418 of dipole antennas 415 for focused microwave transmission in an implementation. In operational scenario 400, antennas 415 are positioned to surround spherical volume 419 to generate a microwave hot spot within volume 419. For example, a computing device, such as computing device 130, may cause a controller, such as controller 120, to drive antennas 415 to emit microwaves according to an optimal antenna configuration which specifies the orientation and power parameters for each of antennas 415 to generate a hot spot at a specified location in volume 419.

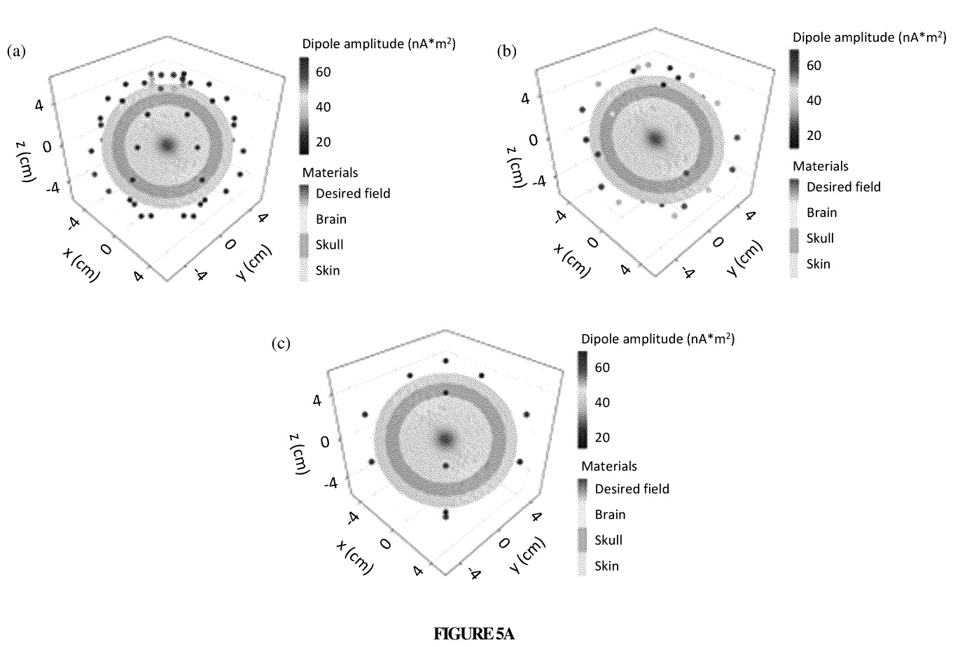

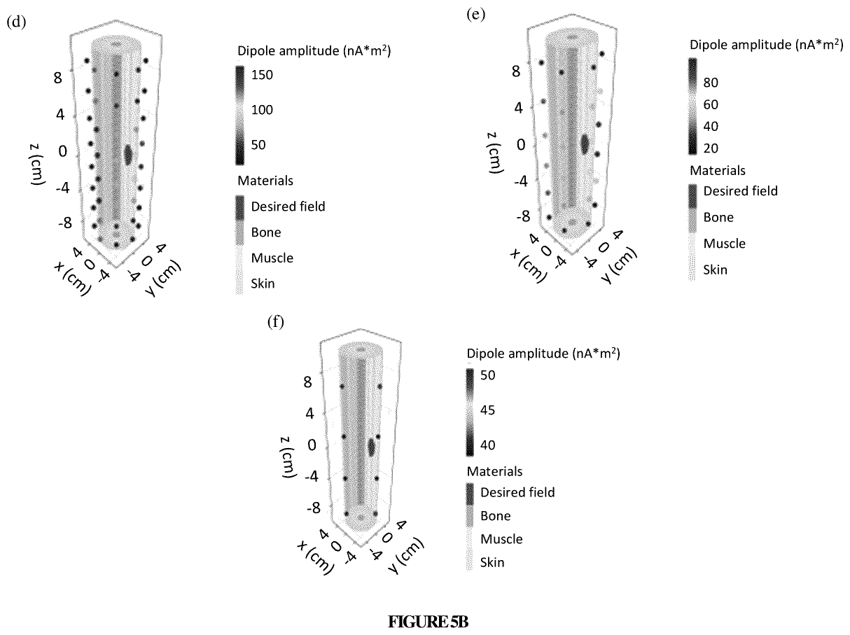

FIGS. 5A and 5B illustrate implementations for focused microwave transmission for three-dimensional antenna arrays in spherical and cylindrical geometries with varying numbers of antennas and according to different transmission intensities for simulated human biological tissue. In FIG. 5A, illustration (a) depicts a 64-antenna configuration for a spherical geometry; illustration (b) depicts a 32-antenna configuration for a spherical geometry; and illustration (c) depicts a 16-antenna configuration for a spherical geometry. In FIG. 5B, illustration (d) depicts a 64-antenna configuration for a cylindrical geometry; illustration (e) depicts a 32-antenna configuration for a cylindrical geometry; and illustration (f) depicts a 16-antenna configuration for a cylindrical geometry.

In each illustration in FIGS. 5A and 5B, the microwave hot spot or desired field is generated by the microwave emissions of the individual antennas according to an optimal antenna configuration or solution generated by an optimization algorithm. The optimization algorithm receives antenna position information, a treatment or hot spot location, a spatial distribution of material properties, and type of treatment, and computes an antenna configuration to illuminate the target location without minimal or no effect on the surrounding tissue.

FIG. 6 illustrates computing device 601 that is representative of any system or collection of systems in which the various processes, programs, services, and scenarios disclosed herein may be implemented. Examples of computing device 601 include, but are not limited to, desktop and laptop computers, tablet computers, mobile computers, and wearable devices. Examples may also include server computers, web servers, cloud computing platforms, and data center equipment, as well as any other type of physical or virtual server machine, container, and any variation or combination thereof.

Computing device 601 may be implemented as a single apparatus, system, or device or may be implemented in a distributed manner as multiple apparatuses, systems, or devices. Computing device 601 includes, but is not limited to, processing system 602, storage system 603, software 605, communication interface system 607, and user interface system 609 (optional). Processing system 602 is operatively coupled with storage system 603, communication interface system 607, and user interface system 609.

Processing system 602 loads and executes software 605 from storage system 603. Software 605 includes and implements microwave transmission process 606, which is (are) representative of the microwave transmission processes discussed with respect to the preceding Figures, such as process 200. When executed by processing system 602, software 605 directs processing system 602 to operate as described herein for at least the various processes, operational scenarios, and sequences discussed in the foregoing implementations. Computing device 601 may optionally include additional devices, features, or functionality not discussed for purposes of brevity.

Referring still to FIG. 6, processing system 602 may comprise a micro-processor and other circuitry that retrieves and executes software 605 from storage system 603. Processing system 602 may be implemented within a single processing device but may also be distributed across multiple processing devices or sub-systems that cooperate in executing program instructions. Examples of processing system 602 include general purpose central processing units, graphical processing units, application specific processors, and logic devices, as well as any other type of processing device, combinations, or variations thereof.

Storage system 603 may comprise any computer readable storage media readable by processing system 602 and capable of storing software 605. Storage system 603 may include volatile and nonvolatile, removable and non-removable media implemented in any method or technology for storage of information, such as computer readable instructions, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 603 may also include computer readable communication media over which at least some of software 605 may be communicated internally or externally. Storage system 603 may be implemented as a single storage device but may also be implemented across multiple storage devices or sub-systems co-located or distributed relative to each other. Storage system 603 may comprise additional elements, such as a controller, capable of communicating with processing system 602 or possibly other systems.

Software 605 (including microwave transmission process 606) may be implemented in program instructions and among other functions may, when executed by processing system 602, direct processing system 602 to operate as described with respect to the various operational scenarios, sequences, and processes illustrated herein. For example, software 605 may include program instructions for implementing a focused microwave transmission process as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 605 may include additional processes, programs, or components, such as operating system software, virtualization software, or other application software. Software 605 may also comprise firmware or some other form of machine-readable processing instructions executable by processing system 602.

In general, software 605 may, when loaded into processing system 602 and executed, transform a suitable apparatus, system, or device (of which computing device 601 is representative) overall from a general-purpose computing system into a special-purpose computing system customized to support focused microwave transmission in an optimized manner. Indeed, encoding software 605 on storage system 603 may transform the physical structure of storage system 603. The specific transformation of the physical structure may depend on various factors in different implementations of this description. Examples of such factors may include, but are not limited to, the technology used to implement the storage media of storage system 603 and whether the computer-storage media are characterized as primary or secondary storage, as well as other factors.

For example, if the computer readable storage media are implemented as semiconductor-based memory, software 605 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 607 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing device 601 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Indeed, the included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in various ways to form multiple embodiments. As a result, the invention is not limited to the specific embodiments described above, but only by the claims and their equivalents.

What is claimed is:

1. A computing apparatus comprising:
one or more computer readable storage media;
one or more processors operatively coupled with the one or more computer readable storage media; and
program instructions stored on the one or more computer readable storage media that, when executed by the one or more processors, direct the computing apparatus to at least:
identify a location for receiving focused microwave emissions from an array of antennas;
determine an optimal antenna configuration for illuminating the location by the microwave emissions based at least on a configuration of the array; and
cause the antennas to illuminate the location according to the optimal antenna configuration.

2. The computing apparatus of claim 1, wherein the optimal antenna configuration includes emission parameters and orientation information for each antenna of the array of antennas.

3. The computing apparatus of claim 1, wherein to determine the optimal antenna configuration for illuminating the location, the program instructions further direct the computing apparatus to determine the optimal antenna configuration based on a spatial distribution of material properties of a volume that includes the location.

4. The computing apparatus of claim 3, wherein the spatial distribution of material properties of the volume is determined based on imaging data of the volume.

5. The computing apparatus of claim 3, wherein the program instructions further direct the computing apparatus to receive imaging data for the volume and determine the spatial distribution of material properties of the volume based on the imaging data.

6. The computing apparatus of claim 3, wherein the microwave emissions comprise amplitude-modulated microwave emissions.

7. The computing apparatus of claim 1, wherein the program instructions further direct the computing apparatus to determine, in a course of treatment, a second optimal antenna configuration for illuminating a second location in a volume by the microwave emissions and cause the antennas to illuminate the second location during the course of treatment.

8. The computing apparatus of claim 1, wherein the configuration of the array comprises antenna positions in spherical distribution or a cylindrical distribution.

9. A system of operating a system for microwave focusing, wherein the system comprises:
an array of microwave antennas positioned in a spherical or cylindrical configuration, and
a computing apparatus in communication with the array of microwave antennas, wherein the computing apparatus comprises:
one or more computer readable storage media;
one or more processors operatively coupled with the one or more computer readable storage media; and
program instructions stored on the one or more computer readable storage media that, when executed by the one or more processors, direct the computing apparatus to at least:
identify a location for receiving focused microwave emissions from the array of microwave antennas;
determine an optimal antenna configuration for illuminating the location by the microwave emissions based at least on a configuration of the array; and
cause the microwave antennas to illuminate the location according to the optimal antenna configuration.

10. The system of claim 9, wherein the optimal antenna configuration includes emission parameters and orientation information for each antenna of the array of microwave antennas.

11. The system of claim 10, wherein to determine the optimal antenna configuration for illuminating the location, the program instructions direct the computing apparatus to determine the optimal antenna configuration based at least on a spatial distribution of material properties of a volume that includes the location.

12. The system of claim 11, wherein the spatial distribution of material properties of the volume is determined based on imaging data of the volume.

13. The system of claim 11, wherein the program instructions further direct the computing apparatus to receive imaging data for the volume and determine the spatial distribution of material properties of the volume based on the imaging data.

14. One or more computer readable storage media having program instructions stored thereon that, when executed by one or more processors, direct a computing apparatus to at least:
identify a location for receiving focused microwave emissions from an array of antennas;
determine an optimal antenna configuration for illuminating the location by the microwave emissions based at least on a configuration of the array; and
cause the antennas to illuminate the location according to the optimal antenna configuration.

15. The one or more computer readable storage media of claim 14, wherein the optimal antenna configuration includes emission parameters and orientation information for each antenna of the array of antennas.

16. The one or more computer readable storage media of claim 14, wherein to determine the optimal antenna configuration for illuminating the location, the program instructions direct the computing apparatus to determine the optimal antenna configuration based at least on a spatial distribution of material properties of a volume that includes the location.

17. The one or more computer readable storage media of claim 16, wherein the spatial distribution of material properties of the volume is determined based on imaging data of the volume.

18. The one or more computer readable storage media of claim 16, wherein the program instructions further direct the computing apparatus to receive imaging data for the volume and determine the spatial distribution of material properties of the volume based on the imaging data.

19. The one or more computer readable storage media of claim 16, wherein the microwave emissions comprise amplitude-modulated microwave emissions.

20. The one or more computer readable storage media of claim 16, wherein the configuration of the array comprises antenna positions in spherical distribution of at least 32 antennas.
